(19) United States
(12) Patent Application Publication
(10) Pub. No.: US 2003/0161882 A1
(43) Pub. Date: Aug. 28, 2003

(54) Osmotic delivery system

(76) Inventor: Kenneth C. Waterman, East Lyme, CT (US)

Correspondence Address: PFIZER INC., PATENT DEPARTMENT, MS8260-1611, EASTERN POINT ROAD, GROTON, CT, 06340, US

(21) Appl. No.: 10/352,258

(22) Filed: Jan. 27, 2003

Related U.S. Application Data
(60) Provisional application No. 60/353,502, filed on Feb. 1, 2002.

(51) Int. Cl. A61K 31/519; A61K 31/137; A61K 9/22; A61K 9/24

(52) U.S. Cl. 424/468; 514/252.16; 514/651

(57) ABSTRACT

An osmotic pharmaceutical tablet is described which comprises a single-layer compressed core surrounded by a water permeable layer having a passageway. The single-layer core contains (i) a non-ripening drug having a solubility per dose less than about 1 mL⁻¹, (ii) about 2.0% to about 30% by weight of a polyethyleneoxide having a weight-average, molecular weight from about 200,000 to about 7,000,000, (iii) an osmagent, and (iv) an optional disintegrant.

### FIELD OF INVENTION

The present invention relates to a pharmaceutical osmotic delivery system, in particular, a simple osmotic tablet for delivering low-solubility pharmaceutical agents.

### BACKGROUND

The use of oral therapeutic systems having extended release of a drug for effecting a controlled systemic response over time and their advantages over conventional dosage forms such as dispersible tablets and syrups are well-known in the art. Of particular interest are the osmotic systems. The pioneering work for elementary osmotic pumps (also referred to as “simple osmotic systems”) is described by Theeuwes in J. Pharm. Sc., 64(12), 1987-1991 (1975), and in U.S. Pat. Nos. 3,845,770; 3,916,899; 4,077,407; and 4,160,020. The osmotic dispensing device is based on an internal/external osmotic pressure differential (e.g., osmotic pressure gradient across a water-permeable wall against an external fluid). In the simple osmotic system, the device is in the form of a tablet consisting of a solid core surrounded by a water-permeable membrane. Aqueous body fluids enter the system continuously through the water-permeable membrane and dissolve the solid active substance contained within the core. The drug is then released through an orifice in the membrane once sufficient pressure is built up to cause the solution containing the drug to be pushed through the orifice. When the active substance present in the core is able to produce a sufficiently high osmotic pressure of its own or when additives are present to increase the osmotic pressure (i.e., osmagents), the drug is released at a predetermined rate to achieve the desired therapeutic effect. The prerequisite for achieving this effect is a sufficiently high solubility of water-soluble drug such that the amount of water entering the core through the water-permeable membrane is sufficient to dissolve most of the drug in the core. As a result, the drug is delivered from the tablet in a predominantly soluble form.

For drugs that are insoluble or have low-solubility in the fluid environment (e.g., bodily fluids), osmotically controlled delivery of the drug to elicit the desired therapeutic effect is more difficult. For this reason, the simple osmotic systems have been generally considered unsuitable for insoluble or sparingly soluble drugs.

One approach for solving this problem is described in U.S. Pat. No. 4,615,698, which discloses the use of a collapsable water-permeable wall that surrounds the pharmaceutical core. The drug or drug/osmagent may be present alone or in combination with a viscosity-inducing agent. The viscosity-inducing agent acts by increasing the viscosity surrounding the drug in the device and thereby entraining the drug in the exiting fluid. Several different non-ionic water-soluble compounds are listed as suitable viscosity inducing agents. Unlike the earlier osmotic devices, the water-permeable wall collapses as the drug is delivered through an orifice in the wall. The advantage of this system is the nearly complete delivery of the drug from the device. However, to function properly, the outer membrane must be designed such that it does not rupture from the osmotic pressure generated within the core. As a result, finding the proper thickness and elasticity of the membrane for a particular application and then maintaining those properties during manufacture of the device can be difficult. To date, no commercial embodiment using this technology has been realized.

Another approach involves two-compartment systems (also known as “push-pull” systems). See, e.g., U.S. Pat. No. 4,111,202. In a push-pull system, the drug or drug formulation is present in one compartment and water-soluble or water-swellable auxiliaries (e.g. salts, sugars, swellable polymers and hydrogels) for producing an osmotic pressure are present in a second compartment. The two compartments are separated from each other by a flexible partition and sealed externally by a rigid water-permeable membrane. Fluids entering the second compartment cause an increase in volume of the lower compartment, which in turn acts on the expanding flexible partition and expels the contents of the drug compartment from the system. The preparation of push-pull systems is technically complicated. For example, a flexible partition consisting of a material different from that of the water-permeable membrane has to be incorporated into the dosage form. In addition, for sparingly soluble high-dosage drugs (e.g. more than 200 mg dose), a push-pull system would be voluminous thus making its ingestion difficult.

Push-pull systems for sparingly soluble drugs without a partition are disclosed in U.S. Pat. No. 4,327,725. A commercial embodiment of this system is known as GITS (gastrointestinal therapeutic system) and is marketed in commercial products such as Procardia™ XL and Glucotrol™ XL (both available from Pfizer, Inc., New York, N.Y.). The core consists of two layers: one layer containing the drug and a second layer containing an osmotic driving member. A rigid water-permeable layer surrounds the core and contains a passageway in communication with the drug layer only. The osmotic driving member is a swellable polymer or hydrogel (e.g., polyethylene oxide). Absorption of fluid into the system causes the hydrogel in the second layer to expand thus forcing the contents of the drug layer through the passageway. Compared with conventional coated tablets, the preparation of these tablets is complicated. Not only does this system require a more complex bilayer press to tablet, but also, stringent demands are placed on the properties of the two formulations being compressed together to form a cohesive core. In addition, placement of the passageway is critical to the successful delivery of the drug (e.g., the orifice must be in communication with only the drug containing layer). This system is generally limited to doses of active drug or combination of drug and functional additives lower than about 200 mg.

Another approach for delivering sparingly soluble drugs in an osmotic tablet is the addition of a gas generating means to the tablet core. U.S. Pat. Nos. 4,036,228 and 4,265,874 disclose a single layer core containing a limited solubility drug, a gas generating means (e.g., effervescent couple), an osmagent and a surfactant having wetting, solubilizing and foaming properties (e.g., sodium lauryl sulfate). Fluids imbibing through a rigid water-permeable membrane surrounding the core causes the gas-generating means to produce a gas which creates a pressure sufficient to expel the drug through an orifice in the membrane. Since this dosage form depends on reaction of acid with internal contents to produce a gas, it only maintains the pressure under the acidic conditions present in the stomach. This makes it unsuitable for extended duration delivery since the residence time in the stomach is limited, especially in the fasted state. In addition, the gas generation will depend on individual properties such as what foods and drinks were eaten before and immediately after swallowing the tablet. Providing a tablet sufficiently impermeable to the gas yet permeable to the liquid can also be challenging to produce.

Numerous patents have issued which focus on increasing the solubility of specific sparingly soluble drugs in an osmotic system. For example, U.S. Pat. Nos. 4,610,686 and 4,732,915 disclose the addition of organic acids to increase the solubility of Haloperidol and U.S. Pat. No. 6,224,907B1 discloses the addition of an alkalinizing agent as a solubility enhancer for a leukatriene-receptor antagonist. The success of this approach in enabling elementary osmotic pump type systems is dependent upon the basicity or acidity of the drug being delivered. For many drugs, solubilizing strategies will still not allow sufficient solubility to enable elementary osmotic pumps, or will cause other complications. For example, solubilizing additives will lead to more material in the drug core thereby reducing the amount of drug deliverable by this technology. In other cases, solubilizing additives may adversely affect the drug's stability.

U.S. Pat. No. 4,857,336 (Re. 34,990) discloses an oral therapeutic osmotic system for carbamazepine having only one drug compartment. Although carbamazepine has a low solubility in water, the primary problem being addressed was crystal growth (i.e., ripening) of carbamezepine upon storage or when the water encounters the drug. According to the disclosure, the crystal growth of carbamazepine can be inhibited by adding a protective colloid (e.g., hydroxypropylmethyl-cellulose) to the drug formulation in the core. An improvement of this formulation is disclosed in U.S. Pat. No. 5,284,662 which utilizes a mixture of two different hydroxy (C₁-C₄)alkyl celluloses in combination with the crystal habit modifier and a 1:9 to 9:1 ratio of a C₆-sugar and a mono- or di-saccharide to improve the delivery of carbamazepine from the device.

For reviews that summarize the patent literature and compare the various approaches used in osmotic systems, see Verma, R. K., et al., Drug Development and Industrial Pharmacy, 2617, 695-708 (2000) and Santus, G., et al., “Osmotic drug delivery: a review of the patent literature,” Journal of Controlled Release, 35, 1-21 (1995).

Although several different approaches have been tried in an attempt to incorporate insoluble or low-solubility drugs into an effective osmotic system, there still remains a need for improved systems that provide a more predictable formulation for a wider variety of drug classes and a convenient means for manufacture. In particular, the need remains to provide an improved system capable of delivering higher drug doses of low solubility drugs in a convenient overall dosage size.

### SUMMARY

An osmotic pharmaceutical tablet is provided which comprises (a) a single-layer compressed core comprising: (i) a non-ripening drug having a solubility per dose less than about 1 mL⁻¹, (ii) a polyethyleneoxide having a weight-average, molecular weight from about 200,000 to about 7,000,000, and (iii) an osmagent, wherein the polyethyleneoxide is present in the core from about 2.0% to about 35% by weight (preferably from about 3% to about 20%, more preferably from about 3% to about 15%, most preferably from about 3% to about 10%) and the osmagent is present from about 15% to about 70% by weight (preferably from about 30% to about 65%, more preferably from about 35% to about 55%, most preferably from about 40% to about 50%); (b) a water-permeable layer surrounding the core; and at least one passageway within the layer (b) for delivering the drug to a fluid environment surrounding the tablet. In a preferred embodiment, the combination of the non-ripening drug and the osmagent have an average ductility from about 100 to about 200 Mpa, an average tensile strength from about 0.8 to about 2.0 Mpa, and an average brittle fracture index less than about 0.2. The single-layer core may optionally include a disintegrant (preferably, a non-swelling, non-gelling disintegrant), a bioavailability enhancing additive, and/or a pharmaceutically acceptable excipient, carrier or diluent. The non-ripening drug may be a non-crystalline drug, a crystalline drug, or a drug particle comprising a non-crystalline or crystalline drug and an excipient.

In a preferred embodiment of the present invention, the single-layer compressed core of the osmotic tablet described above comprises a non-ripening drug selected from the group consisting of [2-(3,4-dichlorophenoxy)-5-fluorobenzyl]-methylamine hydrochloride (preferably, in the presence of tartaric acid), sildenafil citrate (preferably, in the presence of ascorbic acid), sertraline hydrochloride, and ziprasidone hydrochloride.

### Definitions

As used herein, the term “non-ripening” is defined as those pharmaceutical agents that are either (i) a non-crystalline drug form (e.g., amorphous drug or drug-excipient solid solutions), or (ii) a crystalline drug form (e.g., polymorphic or hydrate form) having an average particle size in the dosage form such that the average particle size does not increase significantly in size upon contact with moisture (either from storage under normal storage conditions or during operation of the device) in the absence of a protective colloid or crystal-habit modifier. The term “crystal-habit modifier” or “protective colloid” refers to excipients either in a separate phase from the drug particles (powder mixture) or adsorbed to the particle surface which function to prevent crystal growth. A “significant particle size change” is one that hinders the performance of the drug in vivo, for example, by affecting its dissolution rate or ability to be delivered from the dosage form resulting in an at least about 20% decrease in bioavailability (as indicated by the area under the curve (AUC) in a pharmacokinetic plot). An example of the crystal growth (ripening) process with pharmaceutical solids can be found in H. Weiss, Pharmazie, 32, 624-625 (1977). For comparative purposes, an example of a “ripening” drug is anhydrous carbamazepine, which grows long needles when contacted with moisture (as it forms the dihydrate). In the case of anhydrous carbamazepine, ripening in the absence of stabilizing colloids hinders both its absorption in vivo and its delivery from an osmotic device.

The term “limited-solubility” refers to those pharmaceutical agents having a solubility less than about 40 mg/mL at a physiologically relevant pH (e.g., pH 1-8). Included within the meaning of limited-solubility are those drugs that are “substantially water-insoluble” (defined herein as a drug having a minimum water solubility of less than 10 micrograms/mL at a physiologically relevant pH), “sparingly water-soluble” (defined herein as a drug having a minimum aqueous solubility of about 10 micrograms/mL up to about 1 to 2 mg/mL), and “moderately soluble” (defined herein as a drug having a minimum aqueous solubility as high as about 20 to 40 mg/mL).

The term “limited-solubility per dose” refers to active pharmaceutical agents having solubilities divided by their doses of less than about 1 mL⁻¹.

The term “osmagent” or “osmotic agent” refers to any agent that creates a driving force for transport of water from the environment of use into the core of the osmotic device.

The term “drug” refers to a pharmaceutically active ingredient(s), a pharmaceutically acceptable salt thereof, a solvate (including hydrate) of the active ingredient or salt, or a prodrug of the active ingredient, salt or solvate and any pharmaceutical composition formulated to elicit a therapeutic effect in a human or animal.

The term “bioavailability enhancing additive” or “bioavailability enhancer” refers to an additive known in the art to increase bioavailability (e.g., solubilizing agents, additives that increase drug permeability in the GI tract, enzyme inhibitors, and the like).

The term “disintegrant” refers to a substance that facilitates the breakup of a compressed tablet when exposed to water. Disintegrants are described more completely in standard pharmaceutical textbooks such as Remington: The Science and Practice of Pharmacy, Vol. II, pg. 1619, Mack Publishing; Easton, Pa., (1995).

### DETAILED DESCRIPTION

The present invention provides an osmotic pharmaceutical tablet that is capable of delivering limited-solubility drugs without the aid of a separate swellable layer or compartment to force the drug from the device. In addition, the present invention allows one to formulate a dosage form to give a high dose that is smaller in size for easy ingestion. In its simplest form, the osmotic tablet of the present invention comprises a pharmaceutical single-layer core surrounded by a water-permeable coating having a passageway for delivery of the drug from the core.

The pharmaceutical core comprises a non-ripening drug having a limited solubility per dose (i.e., solubility/dose is less than about 1 mL⁻¹), an osmagent, and a water-soluble polymer that aids in the delivery of the drug from the core without adding significant bulk to the tablet. Preferred polymers are water-soluble polymers that are non-ionic at pH's typically found in the GI tract (e.g., pH values between about 2 and about 8). More preferred water-soluble polymers are polyethyleneoxide polymers having a weight average molecular weight from about 200,000 to about 7,000,000.

### Pharmaceutical Agent or Drug

Any non-ripening drug form may be used in the present invention; however, limited-solubility drugs (i.e., drugs having a minimum aqueous solubility of less than about 40 mg/mL in the fluids imbibed into the osmotic device) that need to be delivered in a high dose are of particular interest. The fluid environment is primarily intended to be the gastrointestinal tract, but could include other biological environments where a therapeutic agent can be used for human or animal treatment. Current technologies can accommodate low solubility drugs if the dose is sufficiently low; however, for high doses, there are currently only a limited number of technologies available. Applicants have found that for a solubility-per-dose less than about 1 mL⁻¹, the present invention provides a beneficial osmotic drug delivery system. When it is desirable to deliver bioavailability enhancers in conjunction with the drug, the present invention enables the delivery of a significant amount of such additives such that the total amount of the drug plus bioavailability enhancing additives can be as high as about 1500 mg (preferably less than about 500 mg).

Virtually any pharmaceutical agent having a solubility/dose less than about 1 mL⁻¹ may be used in the present invention. In addition, the drug may be employed in the form of its pharmaceutically acceptable salt as well as in its anhydrous, hydrated, and solvated form and/or in the form of a prodrug. As discussed above, the drug (in the form used) does not ripen in the osmotic device upon contact with moisture (e.g., moisture contact from storage or in operation). The drug can also include a combination of active agents that act either independently or synergistically to provide one or more therapeutic benefits. It may be desirable to combine the drug with bioavailability enhancing additives that serve to improve the overall effectiveness of the active pharmaceutical agent(s). Suitable bioavailability enhancing additives include solubilizing agents which can increase the drug solubility in the biological environment, materials capable of sustaining supersaturation within the biological environment, pH modifiers, buffers, enzyme inhibitors, permeation enhancers, and the like.

As discussed earlier, a non-ripening drug is defined as those pharmaceutical agents that are either (i) non-crystalline drugs, or (ii) crystalline drugs that do not increase significantly in particle size upon exposure to moisture in the absence of a crystal-habit modifier or protective colloid. A significant particle size change is one that decreases the bioavailability (as indicated by the area under the curve (AUC) in a pharmacokinetic plot) of the drug more than about 20% in vivo. An example of the crystal growth (ripening) process with pharmaceutical solids may be found in H. Weiss, Pharmazie, 32, 624-625 (1977). The pharmaceutical agent(s) may be crystalline, non-crystalline, or a mixture thereof so long as the particle size does not increase significantly in the dosage form such that the performance of the drug is hindered in vivo.

For comparative purposes, an example of a “ripening” drug is carbamazepine. The tendency for crystals to grow upon storage or in the presence of water is typically a property of both the chemical nature of the compound and its particle size. In general, the tendency for crystal growth is inversely proportional to the particle size (i.e., the smaller the particle, the higher the tendency for crystal growth upon storage). Crystal growth can be especially troublesome under conditions where supersaturation temporarily occurs within the dosage form. For example, an anhydrous form of a drug may dissolve and then supersaturate the solution with respect to a more stable hydrate (e.g., small particles of anhydrous crystals of carbamezipine). Without protective colloids, the anhydrous crystals dissolve in water then ripen as large hydrated crystals (see, e.g., U.S. Pat. No. 4,857,336). Another example is when the crystal size is below about 1 μm in diameter. A crystal habit modifier (protective colloid) is typically added which functions by changing the surface properties of the drug particles.

Unlike the crystalline ripening drugs described above, one of the drug forms in the practice of the present invention is a homogeneous, non-crystalline mixture of the drug and excipients where the combination can supersaturate. Since the drug form is not crystalline, additives to sustain supersaturation do not function as protective colloids, and therefore fall within the scope of the present invention. An example of such a drug form is described in U.S. Pub. No.2002/0009494 A1, incorporated herein by reference.

A change in drug absorption with crystal size generally depends on the drug solubility, dose, and permeability through the GI walls. Suitable crystal sizes of the drug generally depend on the size of the passageway(s) present in the osmotic device and to some extent on the tendency for the particles to settle inside the dosage form during operation. Preferably, the average drug particle size in the practice of the present invention remains below about 500 μm, more preferably below about 300 μm, and most preferably less than about 100 μm. As discussed above, if the drug is crystalline, then the particle size is preferably greater than about 1 μm to avoid the need to add a crystal-habit modifier. For a detailed discussion of the effects of particle size on drug dissolution and oral drug absorption see R. J. Hintz and K. C. Johnson, Inter. J. Pharm. 51, 9-17 (1989).

The non-ripening drug can be in any solid form (e.g., crystalline, amorphous, or mixtures thereof). The solid form may also include an excipient as part of the drug particles themselves. Drug-excipient combinations can be prepared by methods such as spray-drying, extrusion, lyophilization or other techniques known by those skilled in the art.

For the drug to be entrained in the extruding fluid as it exits the tablet, settling due to gravity or other forces should be avoided. Both the absolute particle density (versus the density of the entraining medium) and the particle size can effect entrainment and can therefore influence the residual level of drug remaining inside the water-permeable coating (or layer) after 24 hours. For that reason, in some cases, it is preferable to use smaller particle sizes (e.g., less than about 20 μm in diameter) to improve performance. Particle size reduction can be carried out using micronizing methods such as jet milling and rapid precipitation. Preferably the average particle size is less than about 50 μm; more preferably below about 30 μm and still more preferably below about 15 μm.

A preferred drug form is prepared with a process and formulation designed to supersaturate the drug in the use environment. Still more preferred is a drug form designed to maintain supersaturation for a sufficient amount of time in the use environment to allow absorption. For example, a drug that is co-administered with an enteric polymer as described in WO 0147495 A1, EP 1027886 A2, EP 1027885 A2, and U.S. Pub. No.2002/0009494 A1, incorporated herein by reference.

Preferred classes of drugs include antihypertensives, antianxiety agents, antidepressants, barbituates, anticlotting agents, anticonvulsants, blood glucose-lowering agents, decongestants, antihistamines, antitussives, antineoplastics, antiarrhythmic agents (such as β-blockers, calcium channel blockers and digoxin), anti-inflammatories, antipsychotic agents, cognitive enhancers, cholesterol-reducing agents, antiobesity agents, autoimmune disorder agents, anti-impotence agents, antibacterial and antifungal agents, hypnotic agents, anti-Parkinsonism agents, anti-Alzheimer's Disease agents, antibiotics, antiviral agents, and HIV protease inhibitors.

Examples of the above and other classes of drugs and therapeutic agents deliverable by the invention are set forth below. Suitable antihypertensives include prazosin, nifedipine, trimazosin and doxazosin; a suitable blood glucose-lowering agent is glipizide; a suitable anti-impotence agent is sildenafil citrate; suitable antineoplastics include chlorambucil, lomustine and echinomycin; a suitable imidazole-type antineoplastic is tubulazole; suitable anti-inflammatory agents include betamethasone, prednisolone, aspirin, flurbiprofen and (+)-N-{4-[3-(4-fluorophenoxy) phenoxy]-2-cyclopenten-1-yl}-N-hyroxyurea; a suitable barbiturate is phenobarbital; suitable antivirals include acyclovir and virazole; suitable HIV protease inhibitors include saquinavir, ritonavir, indinavir, and nelfinavir; suitable vitamins/nutritional agents include retinol and vitamin E; suitable beta blockers include timolol and nadolol; a suitable emetic is apomorphine; suitable diuretics include chlorthalidone and spironolactone; a suitable anticoagulant is dicumarol; suitable cardiotonics include digoxin and digitoxin; suitable androgens include 17-methyltestosterone and testosterone; a suitable steroidal hypnotic/anesthetic is alfaxalone; suitable anabolic agents include fluoxymesterone and methanstenolone; suitable antidepression agents include sulpiride, fluoxetine, paroxetine, venlafaxine, sertraline, [3,6-dimethyl-2-(2,4,6-trimethyl-phenoxy)-pyridin-4-yl]-( 1 -ethylpropyl)-amine and 3,5-dimethyl-4-(3′-pentoxy)-2-(2′,4′,6′-trimethylphenoxy)pyridine; suitable antibiotics include ampicillin and penicillin G; sitable anti-infectives include benzalkonium chloride and chlorhexidine; suitable coronary vasodilators include nitroglycerin and mioflazine; a suitable hypnotic is etomidate; suitable carbonic anhydrase inhibitors include acetazolamide and chlorzolamide; suitable antifungals include econazole, terconazole, fluconazole, and griseofulvin; suitable anthelmintic agents include thiabendazole and oxfendazole; suitable antihistamines include astemizole, levocabastine, cetirizine and cinnarizine; suitable antipsychotics include fluspirilene, penfluridole, resperidone, and ziprasidone; suitable gastrointestinal agents include loperamide and cisapride; suitable serotonin antagonists include ketanserin and mianserin; a suitable anesthetic is lidocaine; a suitable hypoglycemic agent is acetohexamide; a suitable anti-emetic is dimenhydrinate; a suitable antibacterial is cotrimoxazole; a suitable dopaminergic agent is L-DOPA; suitable anti-Alzheimer's Disease agents are THA and donepezil; a suitable anti-ulcer agent/H2 antagonist is famotidine; suitable sedative/hypnotic agents include chlordiazepoxide and triazolam; a suitable vasodilator is alprostadil; a suitable platelet inhibitor is prostacyclin; suitable ACE inhibitor/antihypertensive agents include enalaprilic acid and lisinopril; suitable tetracycline antibiotics include tetracycline and minocycline; suitable macrolide antibiotics include azithromycin, clarithromycin, erythromycin, vancomycin, and spiramycin; suitable glycogen phosphorylase inhibitors include [R-(R*S*)]-5-chloro-N-[2-hydroxy-3-[methoxymethylamino}-3-oxo-1-(phenylmethyl) propyl]propyl]-1H-indole-2-carboxamide and 5-chloro-1H-indole-2-carboxylic acid [(1S)-benzyl-3-((3R,4S)-dihydroxypyrrolidin-1-yl-)-(2R)-hydroxy-3-oxypropyl]amide.

Still further examples of drugs deliverable by the present invention are the glucose-lowering drug chlorpropamide, the anti-fungal fluconazole, the anti-hypercholesterolemic atorvastatin calcium, the antipsychotic thiothixene hydrochloride, the anxiolytics hydroxyzine hydrochloride and doxepin hydrochloride, the anti-hypertensive amlodipine besylate, the antiinflammatories iroxicam, valdecoxib and celicoxib, and the antibiotics carbenicillin indanyl sodium, becampicillin hydrochloride, troleandomycin and doxycycline hyclate.

Preferred drugs include [4-oxo-3-(5-trifluoromethyl-benzothiazol-2-ylmethyl)-3,4-dihydro-phthalazin-1-yl]-acetic acid (zopolrestat), 5-chloro-2-oxo-3-(thiophene-2-carbonyl)-2,3-dihydro-indole-1-carboxylic acid amide (tenidap), mesylate salt of 7-(6-amino-3-aza-bicyclo[3.1.0]hex-3-yl)-1-(2,4-difluoro-phenyl)-6-fluoro-4-oxo-1,4-dihydro-[1,8]naphthyridine-3-carboxylic acid (trovafloxacin mesylate), 2-(3-benzyl-4-hydroxy-chroman-7-yl)-4-trifluoromethyl-benzoic acid and the ethylenediamine salt thereof, [4-(3,4-dichloro-phenyl)-1,2,3,4-tetrahydro-naphthalen-1-yl]-methyl-amine hydrochloride (sertraline hydrochloride; described in U.S. Pat. Nos. 4,536,518 and 5,248,699), L-lysine salt of 6-chloro-5-fluoro-2-oxo-3-(thiophene-2-carbonyl)-2,3-dihydro-indole-1-carboxylic acid amide, mesylate salt of 6-{2-[4-(4-fluoro-phenyl)-4-hydroxy-piperidin-1-yl]-1-hydroxy-propyl}-3,4-dihydro-1H-quinolin-2-one, mesylate salt of 7-{6-[2-(2-amino-propionylamino)-propionylamino]-3-aza-bicyclo[3.1.0]hex-3-yl}-1-(2,4-difluoro-phenyl)-6-fluoro-4-oxo-1,4-dihydro-[1,8]naphthyridine-3-carboxylic acid, 2-(6-chloro-9H-carbazol-2-yl)-propionic acid (carprofen), monohydrate of 5-(2-(4-(1,2-benzisothiazol-3-yl)-1-piperazinyl)-ethyl)-4-chloro-1,3-dihydro-2H-indol-2-one hydrochloride (ziprasidone hydrochloride; described in U.S. Pat. Nos. 4,831,031 and 5,312,925), 1-[3-(6,7-dihydro-1-methyl-7-oxo-3-propyl- 1 H-pyrazolo[4,3-d]pyrimidin-5-yl)-4-ethoxyphenyl]sulfonyl]-4-methylpiperazine citrate (sildenafil citrate; described in U.S. Pat. No. 5,250,534), and [2-(3,4-dichlorophenoxy)-5-fluorobenzyl]-methylamine hydrochloride (described in WO 0050380). Each of the references cited above are incorporated herein by reference.

The active pharmaceutical agent is typically present in the core in an amount less than about 80% by weight; more preferably in an amount from about 1 to about 80% by weight, more preferably in amount from about 1 to about 75%.

### Disintegrants

As mentioned earlier, entrainment of the drug particles in the extruding fluid during operation of the device is highly desirable. For the particles to be well entrained, the drug is preferably well dispersed in the fluid before the drug particles have an opportunity to settle in the tablet core. One means of accomplishing this is by adding a disintegrant that serves to break up the compressed core into its particulate components. Examples of standard disintegrants included materials such as sodium starch glycolate (e.g., Explotab™ CLV), microcrystalline cellulose (e.g., Avicel™), microcrystalline silicified cellulose (e.g., ProSolv™) and croscarmellose sodium (e.g., Ac-Di-Sol™), and other disintegrants known to those skilled in the art. Depending upon the particular formulation, some disintegrants work better than others. When used, the disintegrant is present in amounts ranging from about 1-25% of the core composition; more preferably from about 1-15%; still more preferably from about 1-10%.

### Bioavailability Enhancing Additives

Bioavailability enhancing additives include additives known in the art to increase bioavailability, such as solubilizing agents, additives that increase drug permeability in the GI tract, enzyme inhibitors, and the like. Suitable solubilizing additives include cyclodextrins and surfactants. Other additives that function to increase solubility include acidic or basic additives which solubilize a drug by changing the local pH in the GI tract to a pH where the drug solubility is greater than in the native system. Preferred additives are acids that function to both improve the drug solubility in vivo and to increase the osmotic pressure within the dosage form thereby reducing or eliminating the need for additional osmagents. Preferred acids include ascorbic acid, 2-benzenecarboxylic acid, benzoic acid, fumaric acid, citric acid, edetic acid, malic acid, sebacic acid, sorbic acid and tartaric acid. Bioavailability enhancing additives also include materials that inhibit enzymes that either degrade drug or slow absorption by, for example, effecting an efflux mechanism. Another group of bioavailability enhancing additives include materials that enable drug supersaturation in the GI tract. Such additives include enteric polymers as disclosed in Patent application Nos. WO 0147495 A1, EP 1027886 A2, EP 1027885 A2, and U.S. Pub. No.2002/0009494 A1, incorporated herein by reference. Particular preferred polymers of this type include hydroxypropylmethylcellulose acetate succinate (HMPCAS) and cellulose acetate phthalate (CAP).

Because the osmotic tablet of the present invention enables a large amount of active material to be delivered in a relatively small dosage form (up to about 80% actives), it is particularly suited for delivery of bioavailability enhancing additives to improve drug performance in vivo.

### Water-soluble Polymer

Although several polymers have been disclosed in the art for use in an osmotic tablet, Applicants have found that only a small subset of those polymers provides a commercially useful means for drug delivery in a single-layer osmotic system suitable for limited-solubility drugs. Water-soluble polymers are added to keep drug particles suspended inside the dosage form before they can be delivered through the passageway(s) (e.g., an orifice). High viscosity polymers are useful in preventing settling. However, the polymer in combination with the drug is extruded through the passageway(s) under relatively low pressures. At a given extrusion pressure, the extrusion rate typically slows with increased viscosity. Applicants have surprisingly found that certain polymers in combination with the drug particles form high viscosity solutions with water but are still capable of being extruded from the tablets with a relatively low force. In contrast, polymers having a low molecular weight (<about 200,000) do not form sufficiently viscous solutions inside the tablet core to allow complete delivery due to drug settling. Settling of the drug is a problem when tablets are prepared with no polymer added, which leads to poor drug delivery unless the tablet is constantly agitated to keep drug particles from settling inside the core. Settling is also problematic when the drug particles are large and/or of high density such that the rate of settling increases.

Preferred water-soluble polymers for practice of the invention do not interact with the drug. Since many drugs are ionic, non-ionic polymers are preferred. An example of a non-ionic polymer forming solutions having a high viscosity yet still extrudable at low pressures is Polyox™ coagulant grade (high molecular weight polyethyleneoxide, PEO, available from Union Carbide Incorporated; weight-average MW equal to about 5M and a degree of polymerization equal to about 114,000).

In Example 1 of the Examples section below, a comparison of the efficiency of drug delivery is made among a number of water soluble polymers that are commonly used in osmotic devices (e.g., sodium carboxymethylcellulose (NaCMC), xantham gum, and polyethyleneoxide polymers (PEO) available from Union Carbide under the trade name Polyox). Unlike other commonly used water-soluble polymers, the Polyox™ coagulant provided 90% delivery of the drug in 24 hours under a standard test condition.

Preferred PEO polymers for use in the present invention have a weight-average molecular weight from about 200,000 to about 7 million and a degree of polymerization from about 4,500 to about 160,000. Example 2 shows a comparison of low and high molecular weight PEO. The PEO polymer is typically present in the core in an amount from about 2.0% to about 35% by weight, preferably from about 3% to about 20%, more preferably from about 3% to about 15%, most preferably from about 3% to about 10%. Because of the swelling nature of the PEO, Applicants have found that the maximum amount of the polymer in the core is limited by splitting of the coating during water imbibition. In addition, this splitting depends directly on the molecular weight of the polymer. For example, at a molecular weight of 200,000 (weight average), polymer levels of 35% do not generally burst the coating during tablet use, with molecular weights of 5,000,000 (weight average), levels above 15% will lead to an unacceptable percentage of burst coatings.

### The Osmagent

The core of the drug delivery device of the present invention includes an osmagent (or osmotic agent). The osmagent provides the driving force for transport of water from the environment of use into the core of the device. The osmagent is generally present in the core at a concentration from about 15% to about 70% by weight, preferably from about 30% to about 65%, more preferably from about 40% to about 60%, most preferably from about 40% to about 55%. The effect of osmagent level on the drug delivery profile is shown for two PEO's using a constant osmagent (sorbitol) in Example 3. A wide variety of osmagents can provide the osmotic pressure needed to drive the drug from the osmotic device. The following factors have proven to be useful in selecting an osmagent appropriate for use in the present invention:

(1) potential reaction of the osmagent and any osmagent impurities with the drug;

(2) effect of the osmagent on the solubility of the drug in the use environment;

(3) impact of the osmagent solubility on the drug delivery rate; and

(4) mechanical properties of the osmagent.

Preferably, the osmagent does not significantly lower the solubility of the drug in the use environment. This is particularly an issue when the osmagent is a salt. In many cases, salts can depress the solubility of a salt form of a drug by a common ion effect.

When designing a particular delivery rate, the osmagent is selected based on the amount of water flux needed across the semipermeable membrane to achieve the desired osmotic pressure.

Since the osmagent is typically the bulk excipient, the tableting properties of the osmagent are also considered. Typical tableting properties include flow (generally for direct compressed tablets) and mechanical properties. For the practice of the present invention, it was found that the ductility, tensile strength and brittle fracture index (BFI) (described in Hiestand and Smith in Powder Technology, 38, 145 (1984)) are sufficiently indicative of material properties to select among osmagent options by matching these indices appropriately with those for the drug. For some drugs, the binding of the drug to itself is sufficiently high that the osmagent serves to prevent the drug crystals from forming hard granules (during granulation), in which case, using fine grain osmagents is preferred. When the drug mechanical properties are combined with those of the osmagent, the resulting total blend properties determine the tablet-ability of the blend. If the particle sizes of the drug and osmagent are comparable (within about 25%) the blend properties will be a weighted average of the components. For a first approximation, the properties of the average should preferentially fall within the following ranges to achieve good tablets (i.e., tablets with low friability): ductility from about 100 to about 200 MPa; tensile strength from about 0.8 to about 2.0 MPa; and brittle fracture index (BFI) less than about 0.2. In some cases, a binder may be desired to improve the binding properties of the tablet. Suitable binders include hydroxypropylcellulose such as Klucel™ EXF (available from Hercules Incorporated, Aqualon Division, Wilmington, Del.) and hydroxypropylmethylcellulose such as Pharmacoat™ 603 (available from Shin-Etsu Chemical Company, Japan).

In some cases, the osmagent can serve as bioavailability enhancing additive. For example, some acids (e.g., ascorbic acid) can solubilize some drugs in the GI tract as well as provide sufficient osmotic pressure for operation of the device. When this is possible, use of an osmagent as a solublizer (bioavailability enhancing additive) may be preferred since this allows for a maximum dose of active for a given tablet size.

### Dispersing Aids

In the course of developing this dosage form, it was found that for some drugs, the drug delivery was affected by the dissolution medium used for testing. More specifically, for some drugs, the pH of the dissolution medium affected the dosage form performance. This was traced to the ability of the drug to be dispersed in that medium. As such, we have found that certain additives to the dosage form can improve the dispersing ability of the drug in some dissolution media. Examples include dispersing aids (typically low molecular weight polar polymers such as carbomers or poly(vinylalcohols)), surfactants (such as sodium dodecylsulfate) or agents designed to make the pH inside the tablet core independent of the dissolution medium. A preferred example of the latter is to add an acidifying agent such as tartaric acid. When used, the acid is preferably at a level between 1-50% of the core components; more preferably between 1-30% of the core; still more preferably between 1-20% of the core.

### Optional Excipients

The core formulation may optionally include one or more pharmaceutically acceptable excipients, carriers or diluents. Excipients are generally selected to provide good compression profiles under direct compression. For example, a lubricant is typically used in a tablet formulation to prevent the tablet and punches from sticking in the die. Suitable lubricants include slippery solids such as talc, magnesium and calcium stearate, stearic acid, light anhydrous silicic acid, and hydrogenated vegetable oils. A preferred lubricant is magnesium stearate.

Other useful additives include materials such as surface active agents (e.g., cetyl alcohol, glycerol monostearate, and sodium lauryl sulfate (SLS)), adsorptive carriers (e.g., kaolin and bentonite), preservatives, sweeteners, coloring agents, flavoring agents (e.g., citric acid, menthol, glycine or orange powder), stabilizers (e.g., citric acid, sodium citrate or acetic acid), dispersing agents (e.g., hydroxypropylmethylcellulose), binders (e.g., hydroxypropylcellulose) and mixtures thereof. Typically such additives are present at levels below about 10% of the core weight; and for many such additives, they are typically present below about 1% of the core weight.

### Manufacturing Processes

The pharmaceutical core is prepared by methods that are well-known to those skilled in the art. For example, the components of the core are generally mixed together, compressed into a solid form, the core is overcoated with a water-permeable coating, and then, if necessary, a delivery means through the water-permeable coating is provided (e.g., a hole is drilled in the coating to form an orifice). In some instances, the components can be simply mixed together and then compressed directly. However, it may be desirable for some formulations to be granulated by any technique known to those skilled in the art, followed by subsequent compression into a solid form.

The tablet core is generally prepared by standard tableting processes, such as by a rotary tablet press, which are well-known to those skilled in the art.

### Water-permeable Coating

After compression, the tablets are ejected from the die. The tablets are then overcoated with a water-permeable coating using standard procedures well-known to those skilled in the art. The water-permeable coating contains at least one passageway through which the drug is substantially delivered from the device. Preferably, the drug is delivered through the passageway as opposed to delivery primarily via permeation through the coating material itself. The term “passageway” refers to an opening or pore whether made mechanically, by laser drilling, in situ during use or by rupture during use. Preferably, the passageway is provided by laser or mechanical drilling. The water-permeable coating can be applied by any conventional film coating process well known to those skilled in the art (e.g., spray coating in a pan or fluidized bed coating). The water-permeable coating is generally present in an amount ranging from about 3 wt % to about 30 wt %, preferably from about 6 wt % to about 15 wt %, relative to the core weight.

A preferred form of the coating is a water-permeable polymeric membrane. The passageway(s) may be formed either prior to or during use. The thickness of the polymeric membrane generally varies between about 20 μm and about 800 μm, and is preferably in the range of about 100 μm to about 500 μm. The size of the passageway will be determined by the particle size of the drug, the number of passageways in the device, and the desired delivery rate of the drug during operation. A typical passageway has a diameter from about 25 μm to about 2000 μm, preferably from about 300 μm to about 1000 μm, more preferably from about 400 μm to about 900 μm. The passageway(s) may be formed post-coating by mechanical or laser drilling or may be formed in situ by rupture of the coatings. Rupture of the coating may be controlled by intentionally incorporating a relatively small weak portion into the coating. Passageways may also be formed in situ by erosion of a plug of water-soluble material or by rupture of a thinner portion of the coating over an indentation in the core. Multiple holes can be made in the coating.

Coatings may be dense, microporous or “asymmetric,” having a dense region supported by a thick porous region such as those disclosed in U.S. Pat. Nos. 5,612,059 and 5,698,220, both of which are incorporated herein by reference. When the coating is dense, the coating is composed of a water-permeable material. When the coating is porous, it may be composed of either a water-permeable or a water-impermeable material. When the coating is composed of a porous water-impermeable material, water permeates through the pores of the coating as either a liquid or a vapor.

Examples of osmotic devices that utilize such dense coatings include U.S. Pat. Nos. 3,995,631 and 3,845,770, both of which are incorporated herein by reference. The dense coatings are permeable to the external fluid such as water and may be composed of any of the materials mentioned in these patents as well as other water-permeable polymers known in the art.

The membranes may also be porous as disclosed in U.S. Pat. Nos. 5,654,005 and 5,458,887 or even be formed from water-resistant polymers. U.S. Pat. No. 5,120,548 describes another suitable process for forming coatings from a mixture of a water-insoluble polymer and a leachable water-soluble additive, incorporated herein by reference. The porous membranes may also be formed by the addition of pore-formers as disclosed in U.S. Pat. No. 4,612,008. All of the references cited above are incorporated herein by reference.

In addition, vapor-permeable coatings may even be formed from extremely hydrophobic materials such as polyethylene or polyvinylidenefluoride that, when dense, are essentially water-impermeable, so long as such coatings are porous.

Materials useful in forming the coating include various grades of acrylics, vinyls, ethers, polyamides, polyesters and cellulosic derivatives that are water-permeable and water-insoluble at physiologically relevant pH's, or are susceptible to being rendered water-insoluble by chemical alteration such as by crosslinking.

Specific examples of suitable polymers (or crosslinked versions) useful in forming the coating include plasticized, unplasticized and reinforced cellulose acetate (CA), cellulose diacetate, cellulose triacetate, CA propionate, cellulose nitrate, celluloseacetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta-glucan acetate, beta glucan triacetate, acetaldehyde dimethyl acetate, triacetate of locust bean gum, hydroxlated ethylene-vinylacetate, ethyl cellulose (EC), polyethylene glycol (PEG), polypropylene glycol (PPG), PEG/PPG copolymers, polyvinylpyrrolidone (PVP), hydroxyethyl cellulose (HEC), hydroxypropyl cellulose (H PC), carboxymethyl cellulose (CMC), carboxymethylethyl cellulose (CMEC), hydroxypropylmethyl cellulose (HPMC), hydroxypropylmethyl cellulose propionate (HPMCP), hydroxypropyl methylcellulose acetate succinate (HPMCAS), poly(acrylic) acids and esters and poly(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes and synthetic waxes.

A preferred coating composition comprises a cellulosic polymer, in particular cellulose ethers, cellulose esters and cellulose ester-ethers, i.e., cellulosic derivatives having a mixture of ester and ether substituents, such as HPMCP.

Another preferred class of coating materials are poly(acrylic) acids and esters, poly(methacrylic) acids and esters, and copolymers thereof.

A more preferred coating composition comprises cellulose acetate. Preferred cellulose acetates are those with acetyl contents between 35% and 45% and number-average molecular weights (MWₙ) between 30,000 and 70,000. An even more preferred coating comprises a cellulosic polymer and PEG. A most preferred coating comprises cellulose acetate and PEG. A preferred PEG has a weight-average molecular weight from about 2000 to about 5000; more preferred between 3000 and 4000.

The coating process is conducted in conventional fashion, typically by dissolving the coating material in a solvent and then coating by dipping, spray-coating or preferably by pan-coating. A preferred coating solution contains 5 to 15 weight percent polymer. Typical solvents useful with the cellulosic polymers mentioned above include acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, and mixtures thereof. The use of water based latex or pseudo-latex dispersions are also possible for the coating. Such coatings are preferred due to the manufacturing advantages of avoiding organic solvents and potential environmental challenges therein. Pore-formers and non-solvents (such as water, glycerol and ethanol) or plasticizers (such as diethyl phthalate and triacetin) may also be added in any amount as long as the polymer remains soluble at the spray temperature. Pore-formers and their use in fabricating coatings are described in U.S. Pat. No. 5,612,059, incorporated herein by reference. In general, more water-soluble additives (such as PEG) increase the water-permeability of the coating (and thereby the drug delivery rate) while water insoluble additives (such as triacetin) decrease the rate of drug delivery.

### Additional Coatings

It is often desirable to provide an additional coating or coatings on the inside or outside of the water-permeable coating. Coatings underneath the water-permeable coating are preferably permeable to water. Such coatings can serve to improve adhesion of the water-permeable coating to the tablet core, or to provide a chemical and/or act as a physical barrier between the core and the water-permeable coating. A barrier coating can insulate the core during coating to the water-permeable coating from, for example, the coating solvent or from migration of a plasticizer (e.g., PEG) during storage. External coatings can be cosmetic to help with product identification and marketing, and improve mouth feel and swallowability. Such coatings can also be functional. Examples of such functional coatings include enteric coatings (i.e., coatings designed to dissolve in certain regions in the gastrointestinal tract) and opacifying coatings (designed to block light from reaching a light-sensitive drug). Other product identifying features can also be added to the top of the coating. Examples include, but are not limited to, printing and embossing of identifying information. The additional coating can also contain an active pharmaceutical ingredient, either the same or different from that in the core. This can provide for combination drug delivery and/or allow for specific pharmacokinetics (e.g., pulsatile). Such a coating can be film coated with an appropriate binder onto the tablet.

In addition, active material can be compression coated onto the tablet surface. In many cases, this compression coating can be facilitated by use of a compressible film coat as disclosed in co-pending U.S. Provisional Patent Application No. 60/275889 filed Mar. 14, 2001, and incorporated herein by reference. The compressible coating preferably comprises a gum-based resin (e.g., polyvinyl acetate resin, preferably a polyvinylacetate having a weight average molecular weight from about 2,000 to about 20,000, more preferably from about 10,000 to about 15,000) and a plasticizer. The plasticizer is preferably a water-soluble plasticizer (e.g., polyethyleneglycol). The active material is generally compressed onto the compressible coating in the form of a powder.

### Packaging

The osmotic tablets may be packaged in a variety of ways. Generally, an article for distribution includes a container which holds the osmotic tablets. Suitable containers are well-known to those skilled in the art and include materials such as bottles (plastic and glass), plastic bags, foil packs, and the like. The container may also include a tamper-proof assemblage to prevent indiscreet access to the contents of the package and a means for removing moisture and/or oxygen (e.g., oxygen absorbers such as D Series FreshPax™ packets available from Multisorb Technologies Inc., Buffalo, N.Y., USA, or Ageless™ and ZPTJ™ sachets available from Mitsubishi Gas Corporation, Tokyo, JP). The container typically has deposited thereon a label that describes the contents of the container and any appropriate warnings.

The following Examples illustrate the osmotic systems of the present invention. To exemplify the general concepts of the present invention, specific pharmaceutically active ingredients are used. However, those skilled in the art will appreciate that the particular drugs used are not limiting to the scope of the invention and should not be so construed.

### EXAMPLES

Unless specified otherwise,, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England) or can be made using standard procedures well-known to those skilled in the art. The following materials used in the Examples may be obtained from the corresponding sources listed below:

| | |
|---|---|
| Klucel ™ EXF, EF and HF | Hercules Corporation, Aqualon |
| (hydroxypropylcellulose) | Division, Wilmington, DE |
| Neosorb ™ P110 and 30/60 DC | Rouquette America, Inc. |
| (sorbitol); | Keokuk, IA |
| Magnesium stearate | Mallinckrodt Inc. |
| | Hazelwood, MO |
| Cellulose acetate (398-10) | Eastman Chemicals, |
| 39.8% acetyl content; 10 s | Kingsport, TN |
| falling ball viscosity | |
| Polyethylene glycol (PEG) 3350 | Union Carbide Corp. (subsidiary of |
| Polyox ™ WSR (PEO), | Dow Chemical Co., Midland, MI) |
| Coagulant and N80 grades | |
| Xantham Gum | CP Kelco U.S. Inc. |
| | Chicago, IL |
| NaCMC-7LF | Hercules Corporation, Aqualon ™ |
| NaCMC-7H0F PH | Division, Wilmington, DE |
| (Sodium Carboxymethylcellulose) | |

Sertraline hydrochloride ((1S-cis)-4-(3,4-dichlorophenyl)-1,2,3,4-tetrahydro-N-methyl-1-naphthalenamine hydrochloride) was prepared using the general procedures described in U.S. Pat. Nos. 4,536,518 and 5,248,699, both of which are incorporated herein by reference.

[2-(3,4-Dichlorophenoxy)-5-fluorobenzyl]-methylamine hydrochloride was prepared using the general procedures described in PCT Publication No. WO 0050380 (Example 56).

Unless specified otherwise, tablet cores were prepared using a Manesty™ F-Press (single-punch tablet machine available from Manesty Corporation, Liverpool, UK). Use of such tablet presses is described in Pharmaceutical Dosage Forms: Tablets, Volume 2 (H. A. Leberman, L. Lachman, J. B. Schwartz, Eds.), Marcel Dekker, Inc. New York (1990).

Example 1 illustrates the unexpected benefit of using PEO's in osmotic formulations.

### Example 1

Tests of the effect of different polymers on drug delivery were investigated by preparing tablets by a common procedure. A blend was made by mixing 125.0 g of sertraline HCl, 242.5 g of Neosorb 30/60 DC (sorbitol), 3.5 g of sodium dodecyl sulfate and 25 g of Klucel EXF (HPC). The mixture was passed through a number 18 sieve then blended for 30 minutes using a Turbula™ blender (available from Glen Mills Inc., Clifton, N.J.). 15.84 g of the blend was added to each of 15 bottles. The remaining components were added as indicated in Table I.

TABLE I

| | | | | | Polyox | |
|---|---|---|---|---|---|---|
| Test | Neosorb | NaCMC | Xantham | NaCMC7 | coag- | Polyox |
| Sample | 30/60 DC | 7LF¹ | Gum | H0F PH² | ulant | N80 |
| 1-1 | 3.0 g | 1.0 g | 0.0 g | 0.0 g | 0.0 g | 0.0 g |
| 1-2 | 2.0 g | 2.0 g | 0.0 g | 0.0 g | 0.0 g | 0.0 g |
| 1-3 | 0.0 g | 4.0 g | 0.0 g | 0.0 g | 0.0 g | 0.0 g |
| 1-4 | 3.0 g | 0.0 g | 1.0 g | 0.0 g | 0.0 g | 0.0 g |
| 1-5 | 2.0 g | 0.0 g | 2.0 g | 0.0 g | 0.0 g | 0.0 g |
| 1-6 | 0.0 g | 0.0 g | 4.0 g | 0.0 g | 0.0 g | 0.0 g |
| 1-7 | 3.0 g | 0.0 g | 0.0 g | 1.0 g | 0.0 g | 0.0 g |
| 1-8 | 2.0 g | 0.0 g | 0.0 g | 2.0 g | 0.0 g | 0.0 g |
| 1-9 | 0.0 g | 0.0 g | 0.0 g | 4.0 g | 0.0 g | 0.0 g |
| 1-10 | 3.0 g | 0.0 g | 0.0 g | 0.0 g | 1.0 g | 0.0 g |
| 1-11 | 2.0 g | 0.0 g | 0.0 g | 0.0 g | 2.0 g | 0.0 g |
| 1-12 | 0.0 g | 0.0 g | 0.0 g | 0.0 g | 4.0 g | 0.0 g |
| 1-13 | 3.0 g | 0.0 g | 0.0 g | 0.0 g | 0.0 g | 1.0 g |
| 1-14 | 2.0 g | 0.0 g | 0.0 g | 0.0 g | 0.0 g | 2.0 g |
| 1-15 | 0.0 g | 0.0 g | 0.0 g | 0.0 g | 0.0 g | 4.0 g |

¹Viscosity = 20-50 cps @ 2%
²Viscosity = 1,000-2,800 cps @ 1%

Each bottle was blended in a Turbula™ blender (available from Glen Mills Inc., Clifton, N.J.) for 10 minutes, then 0.20 g of magnesium stearate was added to each bottle. Each blend was Turbula-mixed for an addition 5 minutes. Tablets were prepared using an F-press using 5/16″ SRC tooling (0.8 cm). Tablets were prepared at 300 mg per tablet with hardnesses between 10-12 kP. A coating fluid was prepared by dissolving 35 g of cellulose acetate and 15 g of PEG 3350 in 925 g of acetone and 25 g of water. Tablets were coated on an LDCS-20 coater (available from Vector Corp.) to give a weight gain of between 6 and 8%. One hole was mechanically drilled in each tablet using a 900-μm drill bit. The results of the dissolution experiments (at pH 4.5, acetate buffer) are shown below in Table II (reported as percent dissolved in the dissolution medium as a function of time). Dissolution experiments were carried out in 900 mL of solution per tablet using a CSP Vankel™ dissolution apparatus using paddles at 200 rpm and a temperature of 37° C. Analysis was conducted by HPLC.

TABLE II

| Test Sample | 4 hours | 10 hours | 18 hours | 24 hours |
|---|---|---|---|---|
| 1-1 | 13 | 27 | 44 | 53 |
| 1-2 | 36 | 46 | 54 | 57 |
| 1-3 | 50 | 62 | 66 | 68 |
| 1-4 | 3 | 20 | 52 | 67 |
| 1-5 | 17 | 28 | 71 | 78 |
| 1-6 | 24 | 34 | 40 | 44 |
| 1-7 | 12 | 21 | 36 | 45 |
| 1-8 | 25 | 35 | 46 | 50 |
| 1-9 | 17 | 35 | 53 | 62 |
| 1-10 | 30 | 71 | 94 | 96 |
| 1-11 | 22 | 67 | 83 | 88 |
| 1-12 | 39 | 78 | 90 | 96 |
| 1-13 | 0 | 16 | 98 | 99 |
| 1-14 | 27 | 53 | 87 | 87 |
| 1-15 | 38 | 64 | 98 | 98 |

Example 2 illustrates the importance of high molecular weight PEO vs. low molecular weight PEO.

### Example 2

Tests of the effect of different formulations on drug delivery were investigated by preparing tablets by a common procedure using the formulations outlined in Table III below.

TABLE III

| Component | 2-1 | 2-2 |
|---|---|---|
| [2-(3,4-Dichlorophenoxy)-5-fluorobenzyl]- | 8.4 g | 8.4 g |
| methylamine hydrochloride | | |
| Neosorb ™ 30/60 DC (sorbitol) | 8.4 g | 8.4 g |
| Polyox ™ N80 (PEO MW 200K) | 2.0 g | 0.0 g |
| Polyox ™ coagulant (PEO MW 5M) | 0.0 g | 2.0 g |
| Klucel ™ EXF (HPC) | 1.0 g | 1.0 g |
| magnesium stearate | 0.2 g | 0.2 g |

Blends were prepared by combining each of the components listed above for the two formulations except for the magnesium stearate. The mixtures were hand sieved through a number 18 sieve then blended for 20 minutes using a Turbula™ blender (available from Glen Mills Inc., Clifton, N.J.). The magnesium stearate was added to each blend then each was Turbula-mixed for an additional 5 minutes. Tablets were prepared using an F-press with 5/16″ (8 mm) SRC tooling to give tablets with an average weight of 300 mg. Tablets were then coated with a solution of cellulose acetate, polyethylene glycol 3350, acetone and water with a weight ratio of 4.1/1.9/89.0/5.0. Coatings were carried out using a Vector Hi-Coater LDCS-20 (available from Vector Corporation, Marion, Iowa) to give a total tablet weight corresponding to a 6% weight gain. Each tablet was mechanically drilled with a 0.9 mm drill bit to give one hole through the coating. Analysis was carried out as described in Example 1. The results are shown in Table IV expressed as percent dissolved as a function of time.

TABLE IV

| Test Sample | 6 hrs | 8 hrs | 10 hrs | 14 hrs | 24 hrs |
|---|---|---|---|---|---|
| 2-1 | 20 | 28 | 36 | 63 | 96 |
| 2-2 | 32 | 49 | 65 | 79 | 91 |

Example 3 illustrates the effect of osmagent level on the drug delivery profile using Polyox™ coagulant grade.

### Example 3

Tests of the effect of different osmagent levels on drug delivery were investigated by preparing tablets by a common procedure using the formulations outlined in Table V below.

TABLE V

| Component | 3-1 | 3-2 | 3-3 | 3-4 |
|---|---|---|---|---|
| [2-(3,4-Dichlorophenoxy)-5- | 16.8 g | 14.0 g | 11.2 g | 8.4 g |
| fluorobenzyl]-methylamine | | | | |
| hydrochloride | | | | |
| Neosorb ™ 30/60 DC (sorbitol) | 0.0 g | 2.8 g | 5.6 g | 8.4 g |
| Polyox ™ coagulant | 2.0 g | 2.0 g | 2.0 g | 2.0 g |
| (PEO MW 5M) | | | | |
| Klucel EXF (HPC) | 1.0 g | 1.0 g | 1.0 g | 1.0 g |
| magnesium stearate | 0.2 g | 0.2 g | 0.2 g | 0.2 g |

Blends were prepared by combining each of the components listed above for the four formulations except for the magnesium stearate. The mixtures were hand sieved through a number 18 sieve then blended for 20 minutes using a Turbula™ blender (available from Glen Mills Inc., Clifton, N.J.). The magnesium stearate was added to each blend then each was Turbula-mixed for an additional 5 minutes. Tablets were prepared using an F-press with 5/16″ (8 mm) SRC tooling to give tablets with an average weight of 300 mg. Tablets were then coated with a solution of cellulose acetate, polyethylene glycol 3350, acetone and water with a weight ratio of 4.1/1.9/89.0/5.0. Coatings were carried out using a Vector Hi-Coater LDCS-20 (available from Vector Corporation, Marion, Iowa) to give a total tablet weight corresponding to a 6% weight gain. Each tablet was mechanically drilled with a 0.9 mm drill bit to give one hole through the coating. Analysis was carried out as described in Example 1. The results are shown in Table VI expressed as percent dissolved as a function of time.

TABLE VI

| Test Sample | 6 hrs | 8 hrs | 10 hrs | 14 hrs | 24 hrs |
|---|---|---|---|---|---|
| 3-1 | 7 | 12 | 16 | 25 | 78 |
| 3-2 | 17 | 21 | 32 | 52 | 74 |
| 3-3 | 24 | 35 | 46 | 67 | 85 |
| 3-4 | 32 | 49 | 65 | 79 | 91 |

What is claimed is:

1. An osmotic pharmaceutical tablet comprising
(a) a single-layer compressed core comprising
(i) a non-ripening drug having a solubility per dose less than about 1 mL⁻¹,
(ii) a polyethyleneoxide having a weight-average, molecular weight from about 200,000 to about 7,000,000, and
(iii) an osmagent, wherein said polyethyleneoxide is present in said core from about 2.0% to about 35% by weight and said osmagent is present from about 15% to about 70% by weight;
(b) a water-permeable layer surrounding said core; and
(c) at least one passageway within said layer (b) for delivering said drug to a fluid environment surrounding said tablet.

2. The osmotic tablet of claim 1 wherein said non-ripening drug is non-crystalline.

3. The osmotic tablet of claim 1 wherein said non-ripening drug is crystalline.

4. The osmotic tablet of claim 1 wherein said non-ripening drug is a drug particle comprising a crystalline or non-crystalline drug and an excipient.

5. The osmotic tablet of claim 1 wherein said non-ripening drug is [2-(3,4-dichlorophenoxy)-5-fluorobenzyl]-methylamine hydrochloride.

6. The osmotic tablet of claim 5 wherein said core further comprises tartaric acid.

7. The osmotic tablet of claim 1 wherein said non-ripening drug is sildenafil citrate.

8. The osmotic tablet of claim 7 wherein said osmagent is ascorbic acid.

9. The osmotic tablet of claim 1 wherein said non-ripening drug is sertraline hydrochloride.

10. The osmotic tablet of claim 1 wherein said non-ripening drug is ziprasidone hydrochloride.

11. The osmotic tablet of claim 1 wherein said polyethyleneoxide is present in said core from about 3% to about 20% by weight.

12. The osmotic tablet of claim 1 wherein said polyethyleneoxide is present in said core from about 3% to about 15% by weight.

13. The osmotic tablet of claims 1 wherein said polyethyleneoxide is present in said core from about 3% to about 10% by weight.

14. The osmotic tablet of any one of the preceding claims wherein said osmagent is present in said core from about 30% to about 65% by weight.

15. The osmotic tablet of claim 1 wherein said osmagent is present in said core from about 35% to about 55% by weight.

16. The osmotic tablet of claim 1 wherein said osmagent is present in said core from about 40% to about 50% by weight.

17. The osmotic tablet of claim 1 wherein the combination of said non-ripening drug and said osmagent have an average ductility from about 100 to about 200 Mpa.

18. The osmotic tablet of claim 1 wherein the combination of said non-ripening drug and said osmagent have an average tensile strength from about 0.8 to about 2.0 Mpa.

19. The osmotic tablet of claim 1 wherein the combination of said non-ripening drug and said osmagent have an average brittle fracture index less than about 0.2.

20. The osmotic tablet of any one of the preceding claims wherein said single-layer core further comprises a disintegrant.

21. The osmotic tablet of claim 20 wherein said disintegrant is non-swelling, non-gelling, disintegrant.

22. An osmotic pharmaceutical tablet comprising
(a) a single-layer compressed core consisting essentially of
(i) a non-ripening drug having a solubility per dose less than about 1 mL⁻¹,
(ii) a polyethyleneoxide having a weight-average, molecular weight from about 200,000 to about 7,000,000,
(iii) an osmagent,
(iv) an optional bioavailability enhancing additive, and
(v) an optional pharmaceutically acceptable excipient, carrier or diluent, wherein said polyethyleneoxide is present in said core from about 2.0% to about 35% by weight and said osmagent is present from about 15% to about 70% by weight;
(b) a water-permeable layer surrounding said core; and
(c) at least one passageway within said layer (b) for delivering said drug to a fluid environment surrounding said tablet.
